(19) United States
(12) Patent Application Publication
(10) Pub. No.: US 2011/0028407 A1
(43) Pub. Date: Feb. 3, 2011

(54) ENHANCEMENT OF INNATE RESISTANCE TO INFECTION

(76) Inventors: Mark A. Jutila, Bozeman, MT (US); Kirk J. Lubick, Belgrade, MT (US)

Correspondence Address: COOLEY LLP;ATTN: Patent Group, Suite 1100, 777 - 6th Street, NW, WASHINGTON, DC, 20001, US

(21) Appl. No.: 12/526,325

(22) PCT Filed: Feb. 8, 2008
(86) PCT No.: PCT/US08/53424

§ 371 Date: Oct. 8, 2009

Related U.S. Application Data
(60) Provisional application No. 60/900,326, filed on Feb. 9, 2007.

(51) Int. Cl. A61K 31/4375; A61P 31/04; A61P 31/12; A61P 33/00; A61K 31/7008; A61K 31/397; A61K 38/14; A61K 31/545; A61K 31/43; A61K 31/65; A61K 31/739

(52) U.S. Cl. 514/20.9; 514/306; 514/25; 514/210.09; 514/200; 514/192; 514/154; 514/54

(57) ABSTRACT

The present invention provides compounds and compositions that enhance the innate immune system. The present invention comprises methods of preventing, treating or ameliorating an infectious disease comprising administering said compounds to a subject. The invention also comprises methods of formulation and administration of said compounds.

This application is claims priority to U.S. Provisional Application No. 60/900,326, filed Feb. 9, 2007, which is hereby incorporated by reference in its entirety.

This invention was made with government support under contract U54AI106537 awarded by NIH, contract W9113M-04-1-0010 awarded by the ARMY/SMDC, and contract P20RR020185 awarded by NIH. The government has certain rights in the invention.

### BACKGROUND OF THE INVENTION

A host exposed to microbial pathogens such as viruses, bacteria, and fungi triggers the activation of innate immune responses that initiate early host defense mechanisms as well as invigorate adaptive immune responses involving cytotoxic T cell activity and antibody production (Medzhitov et al. (1998) Semin. Immunol. 10, 351-353). The recognition of pathogenic microbes and the triggering of the innate immune cascade has become the subject of intense research over the past few years. Particular attention has recently focused on the role of the Toll-like receptors (TLRs), which have emerged as key surface molecules responsible for recognizing conserved components of pathogenic microorganisms (referred to as pathogen-associated molecular patterns—PAMPs), such as lipopolysaccharide and CpG DNA (Medzhitov et al. (1998) Semin. Immunol. 10, 351-353). The TLRs were first identified in Drosophila (the fruit fly) and have been demonstrated as playing an important role in fly development as well as in host defense against fungi and gram-positive bacteria (Imler et al. (2002) Curr. Top. Microbiol. Immunol. 270, 53-79).

Toll-like receptors (TLRs) are type I transmembrane proteins known to be involved in innate immunity by recognizing microbial conserved structures. TLRs may also recognize endogenous ligands induced during the inflammatory response. There are eleven TLRs (TLR-1, TLR-2, TLR-3, TLR-4, TLR-5, TLR-6, TLR-7, TLR-8, TLR-9, TLR-10, TLR-11, TLR-12 and TLR-13) (Janeway et al. (2002) Annu Rev Immunol 20, 197-216 and Zhang et al. (2004) Science 303, 1522-1526) that differ in the microbial product that activates the TLR. For example, TLR-1, TLR-2, TLR-4, TLR-5 and TLR-6 recognize or is activated by bacterial products (e.g., Gram positive and Gram negative bacteria). TLR-3, TLR-7 and TLR-8 recognizes viral products (e.g., dsRNA, viral RNA). TLR-9 recognizes bacterial and viral products (e.g., unmethylated CpG motifs frequently found in the genome of bacteria and viruses, but not vertebrates). TLR-2 also recognizes fungal, such as yeast, products (e.g., zymoson, mannan). Plasmacytoid dendritic cells express TLR-3, TLR-7 and TLR-9.

Engagement of a TLR transmits a signal to the cell's nucleus, inducing the cell to begin producing certain proteins such as cytokines, alerting other components of host defenses. Following ligand binding, signaling pathways are initiated through interactions triggered by a Toll/interleukin (IL)-1 receptor (TIR) domain present in the cytosolic region of all TLRs (Akira (2003) J. Biol. Chem. 278, 38105-38108). Many TLRs, including TLR-2, -4, and -5, use a common adaptor protein referred to as MYD88, which contains a TIR domain as well as a death domain (DD). Other adaptor molecules that function similarly to MYD88 (though lack a DD) referred to as TRIF/TICAM, TRAM, and TIRAP/Mal have now been isolated and similarly function in the modulation of TLR activity (Horng et al. (2001) Nat. Immunol. 2, 835-841; Oshiumi et al. (2003) Nat. Immunol. 4, 161-167; Yamamoto et al. (2003) Science, 301, 640-643; Yamamoto et al. (2003) Natl. Immunol. 4, 1144-1150). The resident DD of MYD88 probably facilitates interaction with members of the IL-1 receptor-associated kinase (IRAK) family such as IRAK-1 and -4 which are DD-containing serine-threonine kinases involved in the phosphorylation and activation of TRAF-6 (Cao et al. (1996) Science, 271, 1128-1131; Ishida et al. (1996) J. Biol. Chem. 271, 28745-28748; Muzio et al. (1997) Science 278, 1612-1615; Suzuki et al. (2002) Nature 416, 750-756).

All TLRs trigger common signaling pathways that culminate in the activation of the transcription factors NF-κB as well as the mitogen-activated protein kinases (MAPKs), extracellular signal-regulated kinase (ERK), p38, and c-Jun N-terminal kinase (JNK) (Akira (2003) J. Biol. Chem. 278, 38105-38108). In addition, stimulation of TLR-3 or -4 can activate the transcription factor interferon regulatory factor (IRF)-3, perhaps through TRIF-mediated activation of the noncanonical IκB kinase homologues, IκB kinase-ε (IKKε), and TANK-binding kinase-1 (TBK1), although the exact mechanisms remain to be clarified (Doyle (2002) et al. Immunity 17, 251-263; Fitzgerald et al. (2003) Nat. Immunol. 4, 491-496). Activation of the NF-κB, ERK/JNK, and IRF-3 responsive signaling cascades culminates in the transcriptional stimulation of numerous genes that regulate the innate and adaptive immune responses including the inflammatory response.

Activation of primary innate immune response genes such as IFN-β induces not only anti-viral genes, but also molecules that facilitate innate immune responses involving NK cells, the maturation of macrophages as well as upregulation of chemokines and molecules such as MHC that facilitate T-cell responses. IFN has also been shown to be critically important for the production of antibody responses.

Activation of TLRs results in the activation of professional antigen presenting cells, initiation of acquired immune response, and further elimination of the invasive organism. Among the TLR family members, both TLR-2 and TLR-4 have been shown to recognize bacterial components. Administration of purified LPS has been found to confer protection from subsequent bacterial or viral challenge in various models (Berger et al. (1967) Adv. Pharmacil., 5, 19-26), presumably via stimulation of the innate immune system. Recently, the intrauterine administration of LPS in cattle was shown to facilitate clearance of chronic intrauterine infections associated with infertility (Singh et al. (2000) Anim. Reprod. Sci. 59, 159-166). However, despite the potentially beneficial effects, the pharmacologic use of purified LPS is precluded by extreme toxicity; LPS is highly pyrogenic and promotes systemic inflammatory response syndrome. Thus, there is a need for safe and effective compounds that enhances the innate resistance to infectious diseases in animals.

### SUMMARY OF THE INVENTION

The present invention provides compounds and compositions that enhance the innate immune system. In one embodiment, said compounds activate macrophages. One of the compounds of the invention, securinine, has been shown to be safe for administration in humans. Securinine, as depicted in FIG. 3A, is a GABA receptor antagonist. Although securinine has been used for the stimulation of the CNS, the inventors have surprisingly discovered that securinine also activates macrophages in vivo and in vitro, in the absence of detectable TLR signaling.

Thus, the present invention comprises a method of preventing, treating or ameliorating an infectious disease comprising administering securinine to a subject. In one embodiment, said infectious disease is caused by a bacteria. In another embodiment said bacteria is able to multiply inside a eukaryotic cell. In another embodiment, said bacteria are Coxiella burnetii. In another embodiment, the securinine is administered with an additional compound.

The present invention also comprises a method of activating macrophages in a subject in need thereof by administering to said subject a pharmaceutical composition comprising securinine. In one embodiment, said subject is infected with an intracellular microbe. In another embodiment, said microbe is selected from the group consisting of bacteria, virus and parasite.

The present invention also comprises a method of activating macrophages in a subject in need thereof, comprising administering to said subject a pharmaceutical composition comprising the general formula (I). In one embodiment, said subject is infected with an intracellular microbe. In another embodiment, said microbe is selected from the group consisting of bacteria, virus and parasite. In another embodiment, said bacteria are Coxiella burnetii.

### BRIEF DESCRIPTION OF THE FIGURES

FIG. 1. TLR agonists induce killing of Phase II C. burnetii by human and mouse macrophage cell lines. In panel A, human MonoMac-1 cells infected with C. burnetii (MOI 50:1) for 24 hours were treated with PBS, LPS (1 μg/ml), or FSL-1 (10 μg/ml) and the effect on the number of viable C. burnetii was compared after 96 hours in culture. In panel B, mouse WEHI 164 cells infected with C. burnetii for 24 hours were treated with PBS, LPS (1 μg/ml), or FSL-1 (10 μg/ml) and the effect on the number of viable C. burnetii was compared after 96 hours in culture. Values are means +/−s.d. *p<0.05

FIG. 2. Effect of TLR agonists on clearance of Phase II C. burnetii in vivo. Panel A shows Real Time-PCR quantification of spleen C. burnetii DNA from single animals treated with FSL-1 (8 μg/ml), LPS (100 μg/ml), or carrier/buffer control for 2 hours prior to infection with C. burnetii for 24, 48, 72, or 96 hours. Panel B compares spleen weights and C. burnetii burden determined by Real Time PCR from Balb/c mice injected i.p. with FSL-1 (8, 4, or 1 μg/mouse) or carrier/buffer control 2 hours prior to infection with C. burnetii. Spleens were collected 96 hours after infection. Panel C shows spleen weight, Real Time-PCR quantification of spleen C. burnetii DNA, and relative numbers of viable C. burnetii isolated from the spleens, as determined by the bacLight FACS-based assay, from mice treated with (FSL-1 16 μg) or carrier/buffer control for 24 hours prior to infection with C. burnetii. Again, analyses were done at the 96 hours post-infection time point. *Difference in means significant at a P value<0.05.

FIG. 3. Securinine induces IL-8 release and killing of Phase II C. burnetii by macrophages. Panel A shows the securinine structure. Panel B compares IL-8 production by MonoMac-1 cells treated with securinine (25 μM) or carrier/buffer control. Panel C compares C. burnetii killing by MonoMac-1, WEHI 164, or sheep alveolar macrophages cells treated with securinine (25 μM) or carrier/buffer control (0.5% DMSO). In all infection experiments, macrophages were infected with C. burnetii (MOI 50:1) for 24 hours, washed, and treated with securinine (25 μM) or carrier/buffer control (0.5% DMSO) and cultured for 96 hours. The percent C. burnetii killing was determined by the following formula: 100-(number of viable C. burnetii after compound treatment/number of viable C. burnetii after carrier/buffer control). Values represent means +/−s.d.

FIG. 4. Securinine induces increased cathepsin D protein expression in infected macrophages. WEHI 265 cells were treated with securinine (25 μM) or carrier/buffer control (0.5% DMSO), infected with C. burnetii (MOI 50:1) and incubated overnight. Cells were then stained for cathepsin D and C. burnetii. Panel A compares the percentage of cathepsin D positive cells between securinine and carrier/buffer control treated cells. Panel B shows fluorescent photomicrographs of securinine and carrier/buffer control treated, C. burnetii infected WEHI 265 cells stained with anti-C. burnetii (green) and anti-Cathepsin D (red)antibodies, and DAPI (blue), as described below.

FIG. 5. Securinine-like compounds induce IL-8 release and killing of Phase II C. burnetii in vitro. Panel A illustrates simple structures of the securinine-like compounds. Panel B shows IL-8 production by MonoMac-1 cells treated for 24 hours with securinine-like compounds (4 μM) or carrier/buffer control. Panel C shows C. burnetii killing by MonoMac-1 cells treated with securinine-like compounds (4 μM) normalized to carrier/buffer control. The percent of C. burnetii killing was determined by the following formula: 100-(number of viable C. burnetii after compound treatment/number of viable C. burnetii after carrier/buffer control).

FIG. 6. Securinine pre-treatment increases the clearance of Phase II C. burnetii in vivo. Balb/c mice were treated i.p. with securinine (32 μg) or the carrier/buffer control. After 2 hours, mice were injected i.p. with C. burnetii (1×10⁸) and then sacrificed 96 hours later. Panel A shows the results of experiment #1 in which spleen weights and viable C. burnetii isolated from the spleens using the BacLight kit from five control and five securinine-treated mice are compared. Panel B shows a repeat experiment with spleen weights, viable spleen C. burnetii counts, and Real Time-PCR quantification of C. burnetii DNA from the spleen of five control and five securinine-treated mice. Differences in means, indicated with *, are significant at a P value<0.05.

FIG. 7. Monomac-1 cells (human monocyte cell line) were treated with DMSO/buffer control, 50 μM securinine or 20 μg/ml anisomycin for the indicated times. Lysates were prepared and subjected to Western blot with anti-phospho-p38 map kinase (activated MAPK) or anti-p38 MAPK (total MAPK). Both antibodies were purchased from Cell signaling, Inc. Blots were developed with ECL (GE Healthcare) and exposed to film for autoradiography. Anisomycin was used a positive control.

FIG. 8. Balb/c mice were first infected with 2×10⁴ phase I C. burnetii (Nine Mile Strain) and then 24 hours later treated with difference concentrations of securinine (32 or 128 μg) or DMSO/buffer alone i.p. Four days later, the animals were sacrificed, spleens weighed and spleen bacterial counts determined by PCR. Top panel shows the spleen weight data and bottom panel shows the bacterial counts.

### DETAILED DESCRIPTION

### Compounds of the Invention

The term “agonist,” as used herein, refers to a compound that activates macrophages. The agonist could be a naturally occurring compounds, such as LPS, or synthetic. Upon binding to a macrophage, signaling events are trigged which activate the macrophage and increases its anti-microbial functions.

The term “adjuvant” or “adjuvant of the invention” as used herein refers the compounds that activate the innate immune system. These generally refer to securinine and/or a compound comprising formula (I) and/or a GABA receptor antagonist, see below, or any derivative described herein or subsequently discovered.

The term “antigen” as used herein is defined as a molecule that provokes an immune response. This immune response may involve either antibody production or the activation of specific immunologically-competent cells, or both. The skilled artisan will understand that any macromolecule, including virtually all proteins or peptides, can serve as an antigen. Furthermore, antigens can be derived from recombinant or genomic DNA.

The term an “effective amount” of a compound is an amount of the compound that is sufficient to achieve the intended effect. For example, an effective amount of serurinine, when administered to a subject will enhance the innate immune system, specifically activate macrophages. The effective amount will vary with factors such as the nature of the substance, the route of administration, the formulation comprising the compound, and the size, species, and health condition of the recipient of the compound. Methods to determine the effective amount are known in the art.

The term “activated macrophages” as used herein is a macrophage that has been pulsed the adjuvant of the invention with or without an antigen and capable of activating an immune cell.

The term “vaccine” as used herein is defined as a material used to provoke an immune response after administration of the material to a mammal. The immune response can be a specific or non-specific immune response.

The term “subject” as used herein refers to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

The term “treating” or “treatment” as used herein includes the administration of the compositions, compounds or agents of the present invention to prevent or delay the onset of the symptoms, complications, or biochemical indicia of an infection, alleviating or ameliorating the symptoms or arresting or inhibiting further development of the disease, condition, or disorder (e.g., an infectious disease or inflammation). “Treating” further refers to any indicia of success in the treatment or amelioration or prevention of the disease, condition, or disorder (e.g., an infectious disease or inflammation), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with an infectious disease. Treatment can be prophylactic (to prevent or delay the onset of the disease, or to prevent the manifestation of clinical or subclinical symptoms thereof) or therapeutic suppression or alleviation of symptoms after the manifestation of the disease or condition.

The term “preventing” as used herein refers to preventing the onset of symptoms in a subject that can be at increased risk of an infectious disease or inhibiting the symptoms of an infectious disease.

An “infectious disease” as used herein, refers to a disorder arising from the invasion of a host, superficially, locally, or systemically, by an infectious organism. Infectious organisms include bacteria, viruses, fungi, and parasites. Accordingly, “infectious disease” includes bacterial infections, viral infections, fungal infections and parasitic infections.

The invention comprises a method of preventing, treating or ameliorating an infectious disease comprising administering securinine to a subject. Without being bound by any particular theory, securinine activates macrophages and other immune cells, for instance NK cells and/or T cells, which can respond in an antigen independent fashion. This creates a broad-spectrum resistance to infectious challenge because the immune cells are in active form and are primed to respond to any invading compound or microorganism. As illustrated in the Examples below, the inventors have shown that securinine activates macrophages in vivo and in vitro. The cells and mice treated with securinine more effectively clear a bacterial infection.

Thus, one embodiment of the invention comprises a method of preventing, treating and/or ameliorating a bacterial infection comprising administering securinine to a subject. Examples of bacterial infections that can be treated by administering securinine to a subject are: B. pertussis, Leptospira pomona, S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Campylobacter, Chlamydia, Coccidioides, Corynebacterium (e.g., Corynebacterium diptheriae), E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas. Treponema pollidum, Staphylococcus aureus, Pasteurella haemolytica, Corynebacterium diptheriae toxoid, Meningococcal polysaccharide, Bordetella pertusis, Streptococcus pneumoniae, Clostridium tetani toxoid, and Mycobacterium bovis. In a further embodiment, said method of the invention is intended to treat or prevent anthrax infection and/or any biowarfare infectious agent.

In another embodiment, said bacterial infection is caused by bacteria that are able to multiply inside a eukaryotic cell. Examples of intracellular bacteria infections that can be treated by administering securinine to a subject are: Salmonella enterica serovar typhimurium, Legionella pneumophila, Coxiella burnettii, Francisella tularensis, Mycobacterium tuberculosis, obligate intracellular Chlamydia spp., Listeria monocytogenes, Shigella flexneri, enteroinvasive E. coli and Rickettsia. In a particular embodiment, said bacterial infection is Coxiella burnettii.

Examples of viral infections that can be treated by administering securinine to a subject are: influenza (A and B), corona virus (e.g. SARS), hepatitis viruses A, B, C, D & E3, human immunodeficiency virus (HIV), herpes viruses 1, 2, 6 & 7, cytomegalovirus, varicella zoster, papilloma virus, Epstein Barr virus, para-influenza viruses, adenoviruses, bunya viruses (e.g. hanta virus), coxsakie viruses, picoma viruses, rotaviruses, respiratory syncytial viruses, rhinoviruses, rubella virus, papovavirus, mumps virus, measles virus, polio virus (multiple types), adeno virus (multiple types), parainfluenza virus (multiple types), avian influenza (various types), shipping fever virus, Western and Eastern equine encephalomyelitis, Japanese B. encephalomyelitis, Russian Spring Summer encephalomyelitis, hog cholera virus, Newcastle disease virus, fowl pox, rabies, feline and canine distemper and the like viruses, slow brain viruses, rous sarcoma virus (RSV), Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), Togaviridae (e.g., Rubivirus), and dengue virus. In a further embodiment, said method of the invention is intended to treat or prevent small pox.

Examples of parasitic infections comprise parasites that cause the following infections: leishmaniasis (Leishmania tropica mexicana, Leishmania tropica, Leishmania major, Leishmania aethiopica, Leishmania braziliensis, Leishmania donovani, Leishmania infantum, Leishmania chagasi), trypanosomiasis (Trypanosoma brucei gambiense, Trypanosoma brucei rhodesiense), toxoplasmosis (Toxoplasma gondii), schistosomiasis (Schistosoma haematobium, Schistosoma japonicum, Schistosoma mansoni, Schistosoma mekongi, Schistosoma intercalatum), malaria (Plasmodium virax, Plasmodium falciparium, Plasmodium malariae and Plasmodium ovale) Amebiasis (Entamoeba histolytica), Babesiosis (Babesiosis microti), Cryptosporidiosis (Cryptosporidium parvum), Dientamoebiasis (Dientamoeba fragilis), Giardiasis (Giardia lamblia), Helminthiasis and Trichomonas(Trichomonas vaginalis). The above lists are meant to be illustrative and by no means are meant to limit the invention to those particular bacterial, viral or parasitic infections.

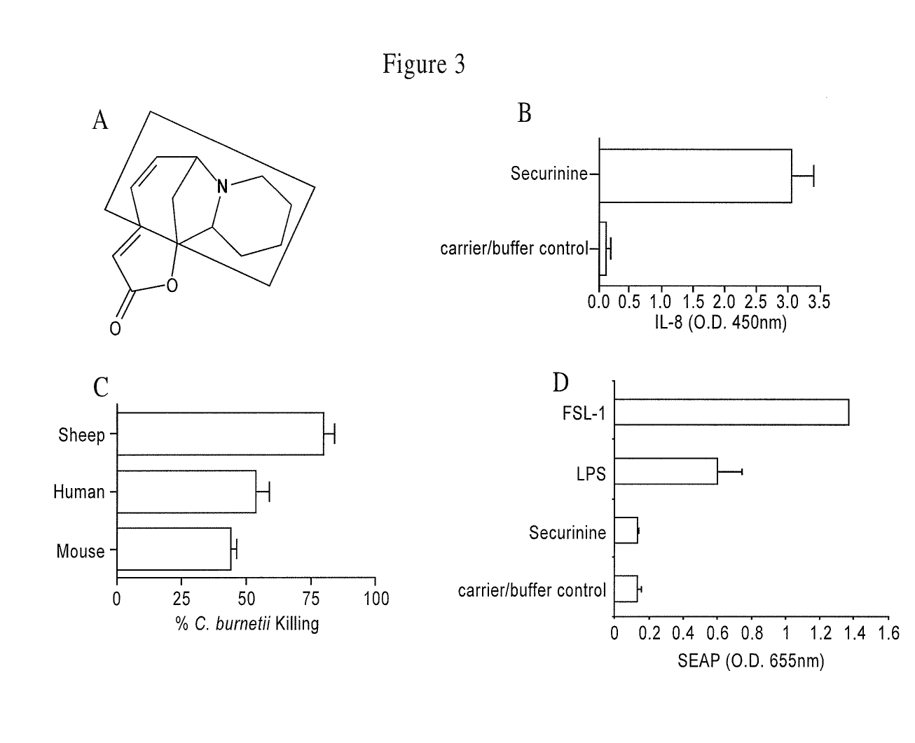

Securinine, a GABA receptor antagonist (FIG. 3A) (11), was identified as a potent inducer of IL-8 secretion in macrophages (FIG. 3B), which has not been previously reported. As shown below, securinine induces the macrophage activation. Thus, one embodiment of the invention comprises a method of activating macrophages in a subject in need thereof by administering to said subject a pharmaceutical composition that comprises securinine. In one embodiment, said subject is infected with an intracellular microbe. In another embodiment, said microbe is selected from the group consisting of a bacteria, virus and parasite (see above for exemplary examples). In another embodiment, said bacteria are Coxiella burnetii. In another embodiment, said subject is administered securinine to prevent an infectious disease. In another embodiment, said securinine composition is administered to said subject orally, intradermally, intranasally, intramusclarly, intraperitoneally, intravenously, or subcutaneously. In another embodiment, the invention comprises a method of enhancing the innate resistance to an infectious disease comprising administering to said subject a pharmaceutical composition that comprises securinine.

GABA receptors, important in neuronal function (7), are expressed by peripheral monocytes and have been shown to affect immune function (1, 10, 26). As shown below, securinine, a GABA antagonist, was able to activate macrophages. Thus, one embodiment of the invention comprises a method of activating macrophages in a subject in need thereof by administering to said subject a pharmaceutical composition that comprises an antagonist of the GABA receptor. In another embodiment, said subject is infected with an intracellular microbe. In another embodiment, said microbe is selected from the group consisting of a bacteria, virus and parasite (see above for exemplary examples). In another embodiment, said bacteria are Coxiella burnetii. In another embodiment, said subject is administered an antagonist of the GABA receptor to prevent an infectious disease. In another embodiment, said an antagonist of the GABA receptor is administered to said subject orally, intradermally, intranasally, intramusclarly, intraperitoneally, intravenously, or subcutaneously. In another embodiment, the invention comprises a method of enhancing the innate resistance to an infectious disease comprising administering to said subject a pharmaceutical composition that comprises an antagonist of the GABA receptor.

The inventors have also discovered additional compounds that activate macrophages. These compounds have the general formula (I):

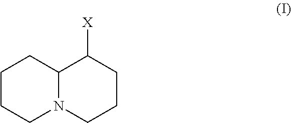

Thus, the invention also comprises a compound that comprises formula (I) and is able to activate macrophages. In another embodiment, the invention comprises a compound that comprises formula (I) and enhances innate resistance to an infectious diseases. In another embodiment, the invention comprises a substituted octahydro quinolizine derivative of formula (I) wherein said formula activates macrophages:

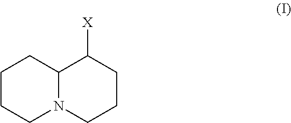

and wherein
X is —NR¹R², —CH₂—NH—C(O)—R³, —CH₂—O—C(O)—R⁴, or —CH₂—OR⁵; wherein
R¹ and R², taken together with the nitrogen atom to which they are shown both attached, form piperidine-2,6-dione, pyrrolidine-2,5-dione, or isoindoline-1,3-dione;
R³ is straight or branched alkyl of 1 to 6 carbon atom;
R⁴ is straight or branched alkyl of 1 to 6 carbon atom, which is unsubstituted or substituted with hydroxyl; and R⁵ is hydrogen, sodium, cyclic alkyl of 5 to 7 carbon atom, or pyrrolidine-2,5-dione.

The invention also comprises a method of activating macrophages in a subject in need thereof by administering to said subject a pharmaceutical composition comprising formula (I). In another embodiment, the invention comprises a method of enhancing the innate resistance to an infectious diseases in a subject in need thereof by administering to said subject a pharmaceutical composition comprising formula (I). In other embodiment, said subject is infected with an intracellular microbe. In another embodiment, said microbe is selected from the group consisting of bacteria, virus and parasite. In another embodiment, said bacteria are Coxiella burnetii.

In another embodiment, said subject is administered a pharmaceutical composition comprising formula (I) to prevent an infectious disease. In another embodiment, said pharmaceutical composition comprises at least one TLR agonist. In another embodiment, said pharmaceutical composition comprises at least one antibiotic. In another embodiment, said pharmaceutical composition comprises at least one additional compound that enhances the immune system. In another embodiment, said subject is administered a pharmaceutical composition comprising securinine.

The invention also comprises a method of preventing, treating or ameliorating a bacterial infection comprising administering to a subject a compound comprising formula (I). As illustrated in the Examples below, the inventors have shown that derivatives of formula (I) activates macrophages and are able to enhance clearance of a bacterial infection. In a particular embodiment, said bacterial infection is Coxiella burnettii.

Another embodiment of the invention comprises a method of preventing, treating or ameliorating a viral infection comprising administering to a subject a compound comprising formula (I).

Another embodiment of the invention comprises a method of preventing, treating or ameliorating a parasitic infection comprising administering to a subject a compound comprising formula (I).

The invention also includes a method for inducing a non-specific innate immune activation and broad-spectrum resistance to microbial challenge using the adjuvants of the invention. The term non-specific innate immune activation as used herein refers to the activation of immune cells, other than B cells. These cells include macrophages, dendritic cells, NK cells, T cells and/or other immune cells, or some combination of these cells that can respond in an antigen independent fashion. A broad-spectrum resistance to infectious challenge is induced because the immune cells are in active form and are primed to respond to any invading compound or microorganism. The cells do not have to be specifically primed against a particular antigen. This is particularly useful in biowarfare and the other circumstances such as traveling to areas with endemic diseases.

The adjuvants of the invention (e.g. securinine and derivatives of formula (I)) can also be formulated and administered with a specific antigen against which one desires an immune response. A microbial antigen, as used herein, is an antigen of a microorganism and includes but is not limited to virus, bacteria, and parasites. Such antigens include the intact organism, natural isolates and fragments or derivatives thereof, and synthetic compounds which are identical to or similar to natural microorganism antigens that induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art. Such combinations will potentate a specific response toward that specific antigen. Such a formulation will be useful as an antigenic formulation or a vaccine against a specific disease. As such, the adjuvants of the invention can be conjugated to a specific antigen. Conjugating molecules to antigens is well known in the art and a person with skill in the art will know what technologies to apply.

### Pharmaceutical Compositions and Methods of Administration

The pharmaceutical compositions useful herein contain a pharmaceutically acceptable carrier, including any suitable diluent or excipient, which includes any pharmaceutical agent that does not itself induce the production of an immune response harmful to the subject receiving the composition, and which may be administered without undue toxicity and securinine. As used herein, the term “pharmaceutically acceptable” means being approved by a regulatory agency of the Federal or a state government or listed in the U.S. Pharmacopia, European Pharmacopia or other generally recognized pharmacopia for use in a subject, more particularly, in humans. These compositions can be useful as a vaccine and/or antigenic compositions for inducing a protective immune response in a subject.

Said pharmaceutical formulations of the invention comprise one or more adjuvants of the invention and a pharmaceutically acceptable carrier or excipient. Pharmaceutically acceptable carriers include but are not limited to saline, buffered saline, dextrose, water, salts, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The invention also provides for a pharmaceutical pack or kit comprising one or more containers filled with one or more of the adjuvant formulation of the invention. In an embodiment, the kit comprises two containers, one containing one or more adjuvants of the invention and the other containing a reconstitution or diluting agent. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the formulation comprising one or more adjuvants of the invention be packaged in a hermetically sealed container such as an ampoule or sachette indicating the quantity of composition. In one embodiment, the adjuvants of the invention is supplied as a liquid, in another embodiment, as a dry sterilized lyophilized powder or water free concentrate in a hermetically sealed container and can be reconstituted, e.g., with water or saline to the appropriate concentration for administration to a subject. In another embodiment, said adjuvants are pressed in to a tablet. The adjuvants of the invention is supplied as a dry sterile lyophilized powder in a hermetically sealed container, or as a tablet, at a unit dosage of about 0.01 mg, about 0.1 mg, about 0.5 mg, about 1 mg, about 5 mg, about 10 mg, about 20 mg, about 25 mg, about 30 mg, about 50 mg, about 100 mg, about 125 mg, about 150 mg, or about 200 mg or higher.

In an alternative embodiment, the adjuvants of the invention is supplied in liquid form in a hermetically sealed container indicating the quantity and concentration of the adjuvant composition. Preferably, the liquid form of the adjuvant composition is supplied in a hermetically sealed container at least about 50 mg/ml, more preferably at least about 100 mg/ml, at least about 200 mg/ml, at least 500 mg/ml, or at least 1 g/ml.

Generally, one or more adjuvants of the invention are administered in an effective amount or quantity sufficient to enhance innate immunity. In another embodiment, one or more adjuvants of the invention are administered in an effective amount or quantity sufficient to activate macrophages. Typically, the dose can be adjusted within this range based on, e.g., age, physical condition, body weight, sex, diet, time of administration, and other clinical factors. The formulation is systemically administered, e.g., by subcutaneous or intramuscular injection using a needle and syringe, or a needle-less injection device, or as a tablet. Alternatively, the formulation is administered intranasally, either by drops, large particle aerosol (greater than about 10 microns), or spray into the upper respiratory tract. In additional embodiments, compositions of the present invention are administered intramuscularly, intravenously, subcutaneously, transdermally or intradermally. Any convenient route, for example by infusion or bolus injection, may administer the compositions by absorption through epithelial or mucocutaneous linings (e.g., oral mucous, colon, conjunctiva, nasopharynx, oropharynx, vagina, urethra, urinary bladder and intestinal mucosa, etc.).

In another embodiment of the invention, said formulation comprising one or more adjuvants of the invention is administered with an additional compound. In one embodiment, said compound is an antibiotic. The antibiotic can be a selected from the group consisting of Aminoglycosides (e.g. Gentamycin, Kanamycin, Neomycin, Streptomycin), Carbapenems (e.g. Ertapenem Imipenem), Chloramphenicol, Fluoroquinolones (e.g. Ciprofloxacin Levofloxacin Norfloxacin), Glycopeptides (e.g. Vancomycin), Lincosamides (e.g. Clindamycin), Macrolides/Ketolides (e.g. Erythromycin, Clarithromycin, Azithromycin), Cephalosporins (e.g. Cefadroxil, Cefaclor, Cefotaxime, Cefepime), Monobactams (e.g. Aztreonam), Penicillins (e.g. Amoxicillin, Ampicillin, Penicillin), and Tetracyclines (e.g. Doxycycline, Minocycline, Tetracycline). One or more antibiotics can be in the formulation of the invention.

In another embodiment, said additional compound is a TLR agonist. Examples of TLR agonists comprise peptidoglycan, RNA, double-stranded RNA, flagellin, unmethylated CpG DNA, profilin, lipoteichoic acids, triacyl lipoproteins and certain viral glycoprotein. In another embodiment, said TLR agonist agonizes TLR-1, TLR-2 TLR-3 TLR-4 TLR-5 TLR-6 TLR-7 TLR-8 TLR-9 TLR-10 TLR-11, TLR-12 and/or TLR-13. In another embodiment, said TLR agonist agonizes TLR-2 and/or TLR-4. In another embodiment, said TLR-2 and/or TLR-4 agonist are selected from the group consisting of lipoteichoic acid, petidoglycan, and lipopolysaccharide.

Said additional compound can be administered simultaneously, e.g. the compound can be formulated with one or more adjuvants of the invention or added to the vial containing said compounds. In another embodiment, said additional compound can be administered consecutively. For example, the adjuvant of the invention can be administered to the subject and the other compound can be added later. The timing can range from a few minutes, to hours, to days. A person of skill in the art can determine the best schedule for such administrations.

Dosages can be determined from animal studies. A non-limiting list of animals includes the guinea pig, Syrian hamster, chinchilla, hedgehog, chicken, rat, mouse and ferret. In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

This invention is further illustrated by the following examples which should not be construed as limiting. The contents of all references, patents and published patent applications cited throughout this application, as well as the Figures, are incorporated herein by reference.

### EXAMPLES

Q-fever, caused by Coxiella burnetii, is a zoonosis that currently lacks an approved vaccine in the U.S., and antibiotics are only partially effective if used early in the course of disease. The disease is acquired primarily through aerosols generated by infected livestock or pets (31), and can manifest as an acute and debilitating illness characterized by malaise, pneumonitis, hepatitis, severe headache and photosensitivity (8, 19). In approximately 5% of cases, Q-fever develops into a chronic, potentially life-threatening disease afflicting the liver (hepatitis) or heart valves (endocarditis) (2). The pathogen is classified as a select agent and is considered a potential bioterrorist weapon. As such, developing new approaches to counter C. burnetii infection is a high priority.

C. burnetii is an obligate intracellular Gram-negative bacterium that infects and resides in macrophages. Highly virulent isolates (Phase I isolates) prevent phagosome/lysosome fusion and induce formation of large replicative vacuoles (LRVs) in which they replicate and survive in infected cells. Less virulent (Phase II) isolates also infect, induce LRV formation and reside in macrophages, but are not as effective at avoiding the killing mechanism of the phagocyte and are eventually cleared in vitro and in vivo (18). Phase I and Phase II isolates both use αvβ3 integrins to gain access into the macrophage, but Phase II isolates are also bound by CR3 (Mac-1, CD11b/CD18), which leads to an increase in the anti-microbicidal activity of the phagocyte and accounts, in part, for differences in virulence (6). Importantly, Phase I isolates are susceptible to the anti-microbicidal actions of macrophages, as long as the macrophage is effectively stimulated. Proper activation leads to phagosome/lysosome fusion, co-localization of cathepsin D, and eventual killing via an NADPH/Oxidant-dependent mechanism (5, 12). Other groups have shown that TLR-2 or TLR-4 agonists induce increased macrophage killing of C. burnetii in vitro and at least for TLR-2, its lack of expression via gene deletion, leads to reduced macrophage killing of the bacterium (15, 32). To date, however, there have been no reports showing that TLR agonists are effective in vivo in increasing resistance to infection with either Phase I or Phase II isolates of C. burnetii.

Tests where conducted for whether adjuvant therapy would be effective in increasing resistance against C. burnetii infection using either TLR-2 or TLR-4 agonists, or other macrophage activating compounds identified during a high throughput drug discovery screening effort. In this initial study, a less virulent Phase II isolates of C. burnetii was used to facilitate testing of multiple agonists. As expected, both TLR-4 and TLR-2 agonists, induced in vitro human and mouse macrophage activation and killing of Phase II C. burnetii. However, the TLR-4 agonist (LPS) had no impact on reducing C. burnetii infection in vivo, and only variable effects were seen with the TLR-2 agonist, FSL-1. In contrast, securinine, which was identified in a screen of 2,000 natural compounds as an activator of human macrophages, was shown to be far more consistent than our best TLR agonist (FSL-1) in inducing increased resistance to C. burnetii infection in vivo. Screening of a drug discovery library based on the securinine structure led to the identification of several synthetic compounds, which exhibited equivalent activity, including in vivo bioactivity, to securinine. Securinine or securinine-like compounds can serve as effective therapeutic adjuvants to increase innate resistance against intracellular pathogens, such as C. burnetii.

### Example 1

### Materials and Methods

Reagents and IL-8 assay. Peptidoglycan (PGN) (Sigma, St. Louis, Mo.), muramyl dipeptide (MDP) (Sigma, St. Louis, Mo.), Pam₃CysSerLys₄ (PAM₃CSK₄) (InvivoGen, San Diego, Calif.), lipoteichoic acid (LTA) (Sigma, St. Louis, Mo.), Pam₃CGDPKHPKSF (FSL-1) (InvivoGen, San Diego, Calif.), and lipopolysaccharide (LPS) (E. coli Sigma, St. Louis, Mo.), 2000 biologically active and structurally diverse natural product compounds (MicroSource Discovery Systems, Gaylordsville, Conn.), and selected TimTec Drug-Like Diversity Compounds (TimTec LLC, Newark, Del.) were tested on MonoMac-1 or U937 cells for induced IL-8 production. Briefly, MonoMac-1 or U937 cells were cultured in cRPMI containing 10% FBS to confluency in a 96 well flat bottom plate. Cells were then stimulated with the test compounds, 20 ng/ml PMA and 0.5 μg/ml Ionomycin (positive control), PBS or DMSO/PBS (0.5%) for 24 hours at 37° C. and 10% CO₂. TLR agonists were resuspended in PBS, whereas the Microsource and TimTec compounds were resuspended in DMSO/PBS (0.5% DMSO). After the 24 hr incubation, supernatant fluid was removed and assayed for the presence of IL-8 by ELISA according to the manufacturer's protocol (R&D Systems, Minneapolis, Minn.).

TLR activation assay. FSL-1 (2 μg/ml), LPS (1 and 0.1 μg/ml E. coli K12, InvivoGen, San Diego, Calif.), and securinine (50 or 25 μM), were tested on THP1-Blue-CD14 cells (InvivoGen, San Diego, Calif.) for TLR agonist activity according to the manufacturer's protocol. THP1-Blue-CD14 cells express TLR-1 to -10, over-express CD14, and are transfected with a reporter plasmid containing secreted embroyonic alkaline phosphatase (SEAP) under the control of both an NF-κB and AP-1 inducible promoter. TLR activation is determined by quantifying SEAP release. Briefly, THP1-Blue-CD14 cells at a concentration of 2×10⁶ cells/ml were cultured in cRPMI containing 10% FBS in addition to glucose (4.5 μg/ml), zeocin (200 μg ml), and blasticidin (10 μg/ml) (all from Invivogen, San Diego, Calif.) followed by PMA (50 ng/ml) treatment for 18 hours. PMA was used to differentiate the THP1 cells to induce expression of TLRs 1-10. Cells were washed to remove residual PMA and the glucose, zeocin, and blasticidin treatment was discontinued. Cells were stimulated with the compounds in cRPMI for 24 hours at 37° C. and 10% CO₂. Supernatant fluid was removed and added to QUANTI-Blue colorimetric assay reagent for 24 hours at 37° C. and 10% CO₂. After 24 hours, samples were read at an O.D. of 655 nm by a VERSAmax tunable microplate reader (Molecular Devices, Sunnyvale, Calif.). All samples were run in quadruplicate, from which averages and standard deviations were determined.

Analysis of peritoneal cells. Female Balb/c mice (6-8 weeks old) acquired from the National Cancer Institute (NCI) (Frederick, Md.) were injected i.p. with different concentrations of FSL-1, PAM₃CSK₄, LPS, securinine, selected TimTec compounds, PBS, or 0.75% DMSO in PBS for 24 hours. Mice were then sacrificed and peritoneal fluid was recovered by injecting 10 ml HBSS into the peritoneum and extracting at least 8 ml for FACS analysis. Cells were washed, counted, and stained with mAbs specific for CD11b [Mac-1α (10 μg/ml), BD Pharmingen, Franklin Lakes, N.J.], Ly6C [Monts-[(10 μg/ml) (16)], Ly6G [RB6-8C5 (10 μg/ml)(16)] or MHC II [AF6-120.1(10 μg/ml), BD Pharmingen, Franklin Lakes, N.J.]. FACS analysis was performed using a BD FACSCalibur and CellQuest software, as previously described (30).

In vitro C. burnetii clearance. MonoMac-1, WEHI 164 (Mouse cell line, ATCC), or WEHI 265 (Mouse cell line, ATCC) were infected with C. burnetii (Phase II Nine Mile strain) at Multiplicities of Infection (MOI) of 50:1 for 24 hours to allow for equal uptake of the bacterium. Cells were washed to remove all non-internalized C. burnetii and stimulated with LPS (10 or 1 μg/ml), FSL-1 (10 μg ml), PAM₃CSK₄ (10 μg/ml), securinine (10-25 μM), TimTec compounds (4.0 nM, PBS, or 0.5% DMSO/PBS for 96 hours. C. burnetii was purified from the cells using differential centrifugation as described by Zamboni (33). Briefly, cells were lysed with H₂O to release C. burnetii and centrifuged at 1000×g for 5 minutes. Supernatant fluid was collected and centrifuged at 14,000×g for 30 minutes to pellet the bacterium. Residual cellular debris was removed by centrifugation at 1,000×g for an additional 5 minutes. C. burnetii was concentrated by centrifugation at 14,000×g for 30 minutes. C. burnetii was then subjected to LIVE/DEAD Baclight Bacterial Viability and Counting Kit (Invitrogen, Carlsbad, Calif.) using FACS to quantify viable C. burnetii.

In vivo C. burnetii clearance studies. Female Balb/c mice (6-8 weeks old) were injected i.p. with FSL-1 (32, 16, 8, or 4 μg/mouse), PAM₃CSK₄ (100, 50, 25, or 5 μg/mouse), LPS (100, 50, 25, or 5 μg/mouse), securinine (32 or 16 μg/mouse), TimTec compounds (32 or 16 μg/mouse), PBS, or 0.75% DMSO/PBS. Mice were infected with an inoculum of 1×10⁸ CFUs of C. burnetii i.p. 2 or 24 hours after compound treatment. Mice were then sacrificed at 24, 48, 72, or 96 hours after infection, and liver, spleen, and peritoneal fluid were collected. Tissues were homogenized using tissue grinders and C. burnetii was purified from the cells using differential centrifugation (as described above). C. burnetii was then used for bacterial viability assays (BacLight) or bacterial DNA was quantified by real time PCR. For the latter, C. burnetii DNA was extracted using the UltraClean™ Microbial DNA Isolation Kit (MO BIO Laboratories, Carlsbad, Calif.). Real Time quantitative PCR was performed using C. burnetii specific Rpos primers (5′-CGCGTTCGTCAAATCCAAATA-3′ and 5′-GACGCCTTCCATTTCCAAAA-3′) designed with Primer Express (Applied Biosystems) as previously described (4). Rpos was quantified by measuring SYBR Green incorporation during real time PCR. PCR reactions were performed in triplicate and data was collected using the GeneAmp 7500 Sequence Detection System (Applied Biosystems).

Immunofluorescence Microscopy. WEHI 265 cells were plated at 5×10⁵ cells/ml and treated with securinine (50 or 25 μM), or carrier/buffer control (0.5% DMSO) for 2 hours, infected with C. burnetii (MOI 50:1) and incubated overnight. Cytospin slide preparations of cells were fixed with 75% ETOH/25% acetone, blocked in PBS containing 10% goat serum, stained with anti-C. burnetii (1/4000) (rabbit anti-C. burnetii polyclonal serum, gift from B. Heinzen, NIH) and anti-Cathepsin D 10 μg/ml (rat anti-mouse) (R&D systems, Minneapolis, Minn.). Anti-C. burnetii was detected by addition of Alexa flour 488 conjugated goat-anti rabbit IgG (Invitrogen, Carlsbad, Calif.) and anti-Cathepsin D was detected by Biotin-SP-conjugated goat anti-rat IgG (Jackson ImmunoResearch,) followed by the addition of Alexa flour 555 conjugated steptavidin (Invitrogen, Carlsbad, Calif.). Slides were cover-slipped using ProLong Gold antifade reagent with DAPI (Invitrogen, Carlsbad, Calif.).

### Example 2

### TLR Agonist Stimulated Cells Kill Phase II C. burnetii In Vitro

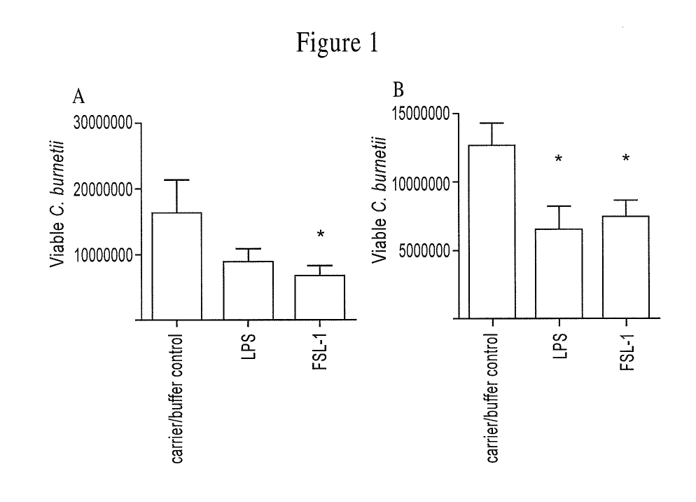

Since TLR-4 and TLR-2 may be important in the clearance of C. burnetii in vivo (15, 32) an experiment was conducted to determine if TLR stimulation of C. burnetii infected human and murine macrophage cell lines would accelerate killing of the bacterium in vitro. Preliminary assays of human macrophage cell lines showed that MonoMac-1 versus U937 cells responded more consistently and robustly to LPS (TLR-4), FSL-1 (TLR-2), PGN (TLR-2 and/or Nod2), LTA (TLR-2), and MDP (Nod2) stimulation, as measured by induced IL-8 release (data not shown). As such, MonoMac-1 cells were used for the infection assays. Next, an experiment was conducted to determine whether C. burnetii could infect MonoMac-1 cells and if so, what affect TLR-2 (FSL-1) and TLR-4 (LPS) stimulation would have on this infection. As shown in FIG. 1A, 96-hours after infection (MOI 50:1), MonoMac-1 cells had contained less C. burnetii if they were treated with FSL-1 or LPS versus PBS alone. This effect was not unique to human cells, in that the mouse WEHI 164 macrophage cell line showed similar results (FIG. 1B).

### Example 3

### FSL-1, But not LPS, Variably Induces Increased Resistance to Phase II C. burnetii Infection In Vivo

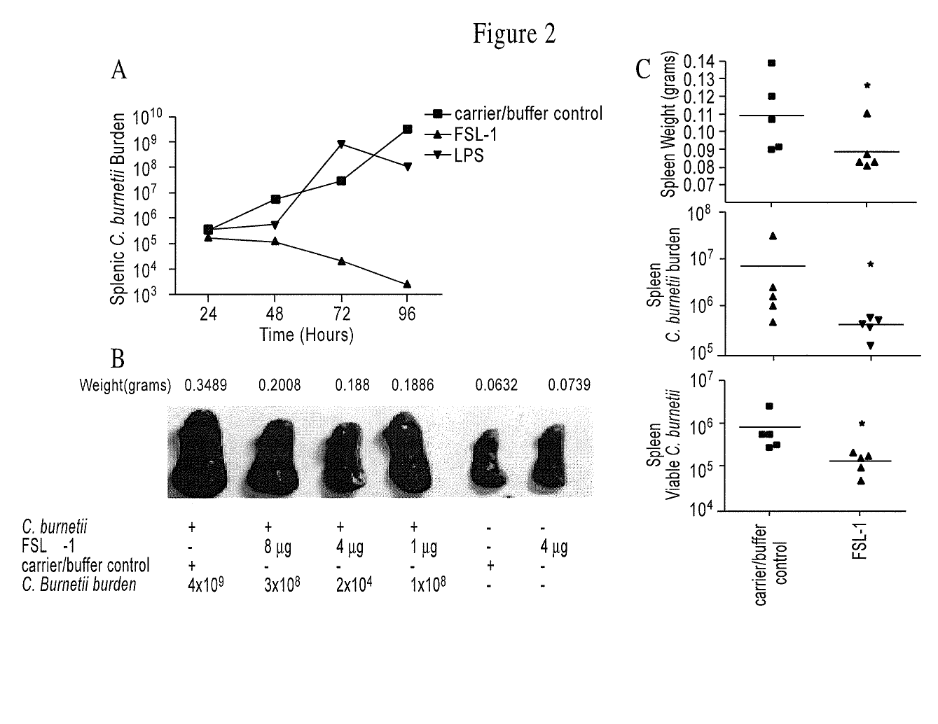

The effect of FSL-1 and LPS on C. burnetii infection in vivo was then examined. First, activity of FSL-1 and LPS was confirmed in vivo by injecting different concentrations of each agonist into the peritoneum of female Balb/c mice and then monitoring the recruitment of inflammatory leukocytes and activation of resident macrophages was test. As expected, each agonist induced neutrophil recruitment, as evidenced by an increase in the percentage of RB6-8C5 positive cells (>30%), and neutrophil and macrophage activation, as evidenced by increased CD11b and Ly6C expression (data not shown). To begin to examine the impact of FSL-1 and LPS on C. burnetii infection, a time course study was done in 4 mice treated i.p. with FSL-1 (8 μg), LPS (100 μg) or buffer alone for 2 hours and then infected i.p. with 1×10⁸ CFUs of C. burnetii. At 24, 48, 72 and 96 hours, the spleen C. burnetii numbers (PCR quantification of bacterial DNA) were compared between the agonist-treated animals and the PBS-treated controls (FIG. 2). C. burnetii levels reduced in the mice treated with FSL-1 compared to buffer control, where as LPS pre-treatment did not have an effect on C. burnetii burden in this experiment (FIG. 2A). A second experiment was done to compare different concentrations of FSL-1 on bacterial counts and the induction of splenomegaly caused by C. burnetii. As shown in FIG. 2B, FSL-1 pretreatment (8 and 4 μg pretreatments) reduced the splenomegaly associated with C. burnetii infection, and 8, 4 and 1 μg FSL-1 treatments all reduced bacterial counts, as determined by PCR (FIG. 2B). Upon additional experimentation, the effects of FSL-1 following only a 2 hr pre-treatment were not seen in all animals: analysis of 18 animals treated with different concentrations of FSL-1 showed a reduction of C. burnetii in only 11 mice (data not shown). No benefit was seen following LPS treatment.

The effect of FSL-1 on C. burnetii infected Balb/c mice was further characterized by testing whether 24 hours pre-treatment might enhance its effectiveness. Both spleen weight (FIG. 2C) and bacterial burden, as determined by PCR (FIG. 2C), were significantly lower (>10-fold) in 5 out of 5 mice treated with FSL-1 for 24 hours prior to infection, as compared to animals treated with buffer alone. Total C. burnetii DNA from both the spleen and liver were lower in the FSL-1 treated mice, as well (Data not shown). As another test, the viable bacterial counts in the spleens of the control and FSL-1 treated animals were analyzed using the FACS-based bacLight assay used in our in vitro analyses. These results also showed that FSL-1 enhanced clearance of C. burnetii (FIG. 2C). Therefore, under the conditions tested here, TLR-2 agonists enhance innate resistance of naïve Balb/c mice to phase II C. burnetii infection, but the timing of TLR agonist treatment was critical.

### Example 4

### Securinine Activates Macrophages and Increases C. burnetii Killing In Vitro

The effects of FSL-1, though variable, prompted us to test other macrophage agonists in the C. burnetii infection model. In a concurrent drug discovery effort, 2,000 natural compounds were screened for macrophage activation activity using IL-8 production in MonoMac-1 cells as a read-out. Securinine, a GABA receptor antagonist (FIG. 3A) (11), was identified as a potent inducer of IL-8 secretion in macrophages (FIG. 3B), which has not been previously reported. As shown in FIG. 3C, securinine also induced killing of C. burnetii in both human and mouse macrophage cell lines. To ensure this effect was not restricted to macrophage tumor cell lines, we tested the effect of securinine on primary alveolar macrophages—the cell that first encounters C. burnetii in a natural infection. Ovine alveolar macrophages were used in these experiments, since sheep are susceptible to an aerosol infection by C. burnetii (17) and their alveolar macrophages are easily obtained by lavage. As shown in FIG. 3C, securinine also induced alveolar macrophage killing of C. burnetii (>80%).

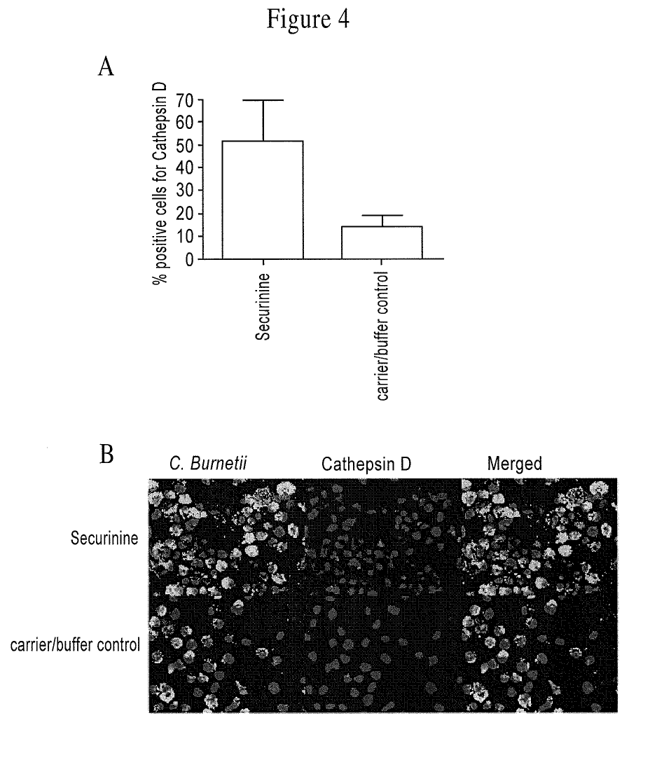

These observations suggested that securinine induced resistance to C. burnetii infection by activating macrophages, thereby increasing their capacity to kill the bacterium. Additional evidence in support of this hypothesis was then sought by 1) testing whether securinine induced macrophage responses necessary for bacterial killing, 2) ensuring that the activity of securinine was not due TLR agonist contaminants, like LPS, and 3) examining whether the effects of securinine were not simply due to toxicity for the bacterium itself or host cell needed for growth of the bacterium. Cathepsin D expression in activated macrophages is a response required for intracellular killing of C. burnetii (12). As shown in FIG. 4, securinine induced cathepsin D expression in infected macrophages compared to buffer control treated cells. The activating effects of securinine were not due to TLR agonist (such as LPS) contamination, since TLR-1 to 10 signaling was not detected in the securinine preparation using the THP1-Blue-CD14/SEAP TLR-1 to 10 assay (FIG. 3D). Finally, to test if the active compounds (FSL-1 and securinine) were directly toxic to the bacterium or the host cell, such that the bacterium could not survive, we incubated C. burnetii or the host macrophages with the compounds and then quantified viable bacterium or macrophages 24 hours later. Neither of the compounds, at concentrations that induced killing in vitro and in vivo, induced direct killing of C. burnetii or the host cells beyond the DMSO/PBS control (data not shown).

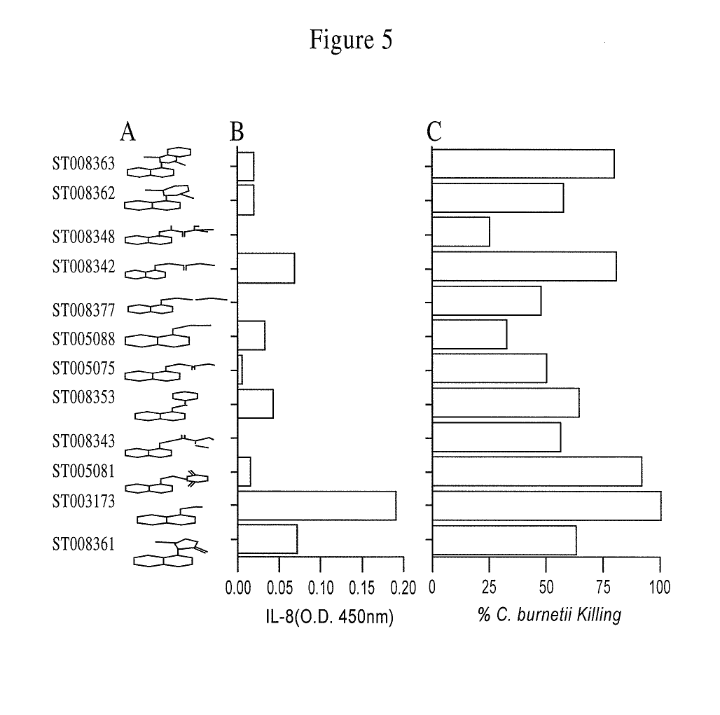

Based on the securinine structure, we identified 18 similar compounds [similar nitrogen containing di-cylic structure (boxed area in FIG. 3A)] in a synthetic compound drug discovery library (TimTec, Inc.). Twelve of the 18 compounds induced IL-8 production by MonoMac-1 cells, though the effective agonist concentration of each compound varied (data not shown). FIGS. 5A and 5B show the impact of the 12 active compounds at a single concentration on IL-8 production by MonoMac-1 cells. All 12 were then tested at the same concentration for their effect on Phase II C. burnetii infection in the same cells. As shown in FIG. 5C, all 12 compounds also reduced C. burnetii infection in vitro to some extent. Of significance, the compound that induced the greatest IL-8 production in this experiment, ST003173, also induced the greatest level of C. burnetii killing (FIG. 5). These results suggest that the nitrogen containing double ring may represent the minimal structure required for the adjuvant activity of this class of adjuvants.

### Example 5

### Securinine Enhances Clearance of Phase II C. burnetii In Vivo

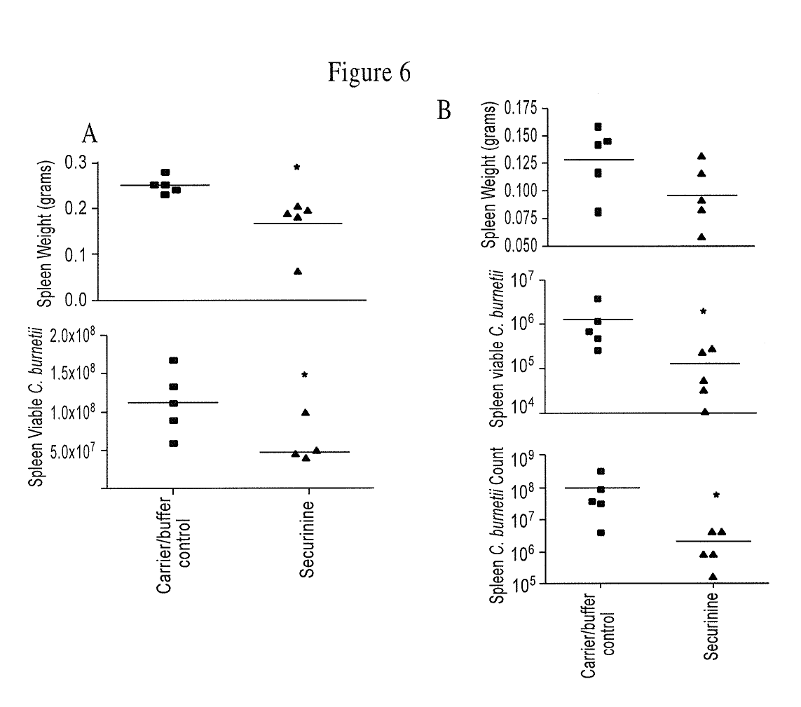

Securinine was then tested for its effect on Phase II C. burnetii infection in vivo. Balb/c mice were treated i.p. with securinine 2 hours prior to infection with 1×10⁸ CFUs of C. burnetii. Based on the peak response to FSL-1 seen in some animals (FIG. 2), we compared spleen weights and C. burnetii burden 96 hours after challenge in the control and securinine treated animals. In this first experiment, we used the most conservative measure of bacterial burden (viable bacterial counts), as determined in our analysis of FSL-1 (FIG. 2). Securinine treated mice had significantly (P value<0.05) lower spleen weights and C. burnetii burden at the 96 hr time point compared to the carrier/buffer (0.75% DMSO/PBS) control (FIG. 6A). To evaluate the consistency of the effect, a second experiment was done using five additional mice and both the viability assay and the PCR assay for bacterial DNA for the analysis of C. burnetii. As shown in FIG. 6B, 10-fold reductions in C. burnetii were detected in the securinine treated animals using both assays (FIG. 6B). Four of the 12 securinine-like compounds, plus securinine, were then tested at two different doses in single animals subsequently infected with C. burnetii. Each, at least at one concentration, had the same effect as securinine in enhancing clearance of C. burnetii from the spleen reducing overall spleen weights (data not shown). Additional animals could not be tested because of limited quantities of the securinine-like compounds.

### Example 6

### Securinine Induces p38Map Kinase (MAPK) Activity in Human Monocytes

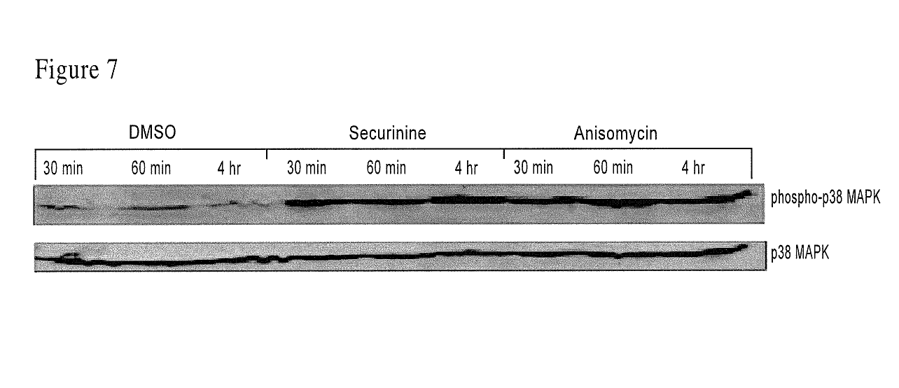

Monomac-1 cells (human monocyte cell line) were treated with DMS/buffer control, 50 μM securinine or 20 μg/ml anisomycin for the indicated times. Lysates were prepared and subjected to Western blot with anti-phospho-p38 map kinase (activated MAPK) or anti-p38 MAPK (total MAPK). Both antibodies were purchased from Cell signaling, Inc. Blots were developed with ECL (GE Healthcare) and exposed to film for autoradiography. Anisomycin was used a positive control. The results are shown on FIG. 7. These results show that securinine induces p38Map kinase activity.

### Example 7

### Securinine Given after Infection Enhances Clearance of Virulent Phase I Coxiella burnetii Infection in Balb/c Mice

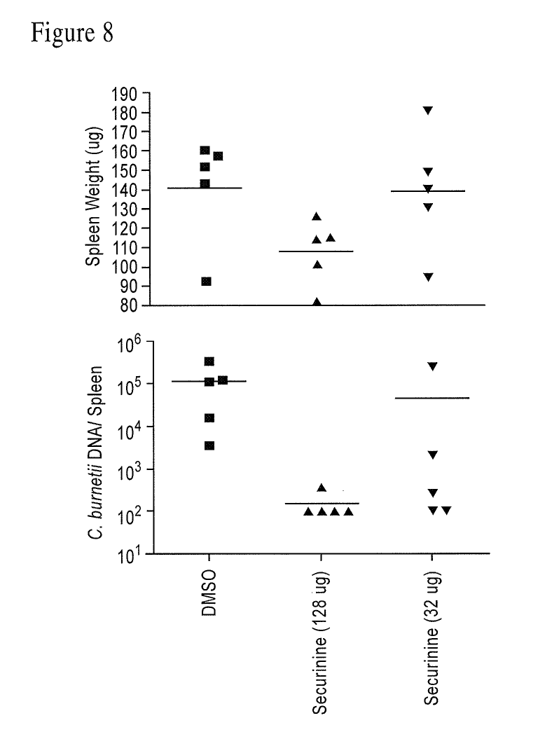

Balb/c mice were first infected with 2×10⁴ phase I C. burnetii (Nine Mile Strain) and then 24 hours later treated with difference concentrations of securinine (32 or 128 μg) or DMSO/buffer alone i.p. Four days later, the animals were sacrificed, spleens weighed and spleen bacterial counts determined by PCR. The results are shown on FIG. 8. The top panel shows the spleen weight data and bottom panel shows the bacterial counts. These data show that mice pretreated with securinine enhances clearance of Coxiella burnetii infection.

### DISCUSSION

Increasing innate immune responses by adjuvant therapy has been shown to be effective in increasing resistance to infectious diseases and represents a complementary approach to vaccines and antibiotics in countering new and reemerging infectious agents (21). TLRs represent targets for most adjuvants in use today, but other innate receptors can also be targeted (9). High throughput screens of natural and synthetic compound libraries were used to identify new innate adjuvants that could be used in vivo and have identified a number of novel macrophage-specific agonists. As shown above, securinine and TLR-2 and TLR-4 agonists where compared for their ability on enhancing innate resistance to C. burnetii infection. As expected, TLR-2 and TLR-4 specific agonists induced macrophage killing of the bacterium in vitro, but were, surprisingly, less effective in vivo. In contrast, securinine and a number of securinine-like compounds from a synthetic drug discovery library induced C. burnetii killing in vitro and in vivo. These results suggest that securinine, or securinine-like compounds, may be effective adjuvants for the innate immune system and aid in increasing resistance or accelerating clearance of intracellular pathogens, such as C. burnetii.

Despite in vitro activity detected with every TLR-2 and TLR-4 agonists tested, in vivo effects were surprisingly poor, consistent with earlier reports (32). This result was not due to lack of activity of the agonists in the animal, since it has been shown that each agonist induced peritoneal macrophage activation and neutrophil recruitment after an i.p. injection. Most striking was the complete lack of effect of LPS on increasing clearance of C. burnetii. This, perhaps, is expected and likely due to the fact that the LPS associated with the bacterium itself induces a maximum amount of TLR-4 signaling, thus, there is no added benefit of pre-treating with another LPS. In contrast, FSL-1 did show positive effects in some animals with a pre-treatment period of only two hours (FIG. 2). The inconsistency of FSL-1 was not simply due to dosing, since different amounts of agonist were tested and greater amounts of agonist did not eliminate the animal-to-animal variability. When the pretreatment times were increased from 2 to 24 hours, the efficiency of FSL-1 in inducing enhanced C. burnetii clearance went from 61% to 100% of the animals, respectively. A variable not examined in depth was the amount of C. burnetii used in the challenge experiments. Large inoculums were required in this model to consistently see spread of the bacterium to the spleen and other organs. We predict that more dramatic results will be obtained when smaller inoculums of C. burnetii are used. This, perhaps, could be done by delivering the bacterium by aerosol into the lung (its normal route of infection), which is currently being investigated.

In contrast to the TLR agonists, securinine given only 2 hours prior to C. burnetii challenge consistently enhanced clearance of the bacterium. This was seen in the spleen, liver and peritoneal cavity and was confirmed using two different assays to measure the bacterial burden in these tissues. The in vivo activity of securinine correlated with its capacity to activate macrophages, as evidenced by increased IL-8 production in vitro. Securinine also induced upregulation of important anti-microbial activities of the macrophage necessary for killing C. burnetii, such as cathepsin D production. Activity was not restricted to securinine, since 12 synthetic compounds with similar structures displayed similar activity in vitro, and four were shown to induce enhanced clearance of C. burnetii in vivo. To date, our searches of the literature suggest this is the first report to demonstrate the adjuvant activity of securinine and securinine-like compounds.

Securinine, a plant alkaloid, is an antagonist of the GABA receptor (3). GABA receptors, important in neuronal function (7), are expressed by peripheral monocytes and have been shown to affect immune function (1, 10, 26). Specifically, GABA receptor agonists are thought to suppress lymphocyte cytokine production and proliferation and ROS production by neutrophils (28, 29). As shown above, an antagonist of the GABA receptor drives an activating signal in macrophages, leading to C. burnetii killing. Securinine does not appear to have activity for TLRs 1-10, nor is it contaminated with TLR agonists. A variety of plant alkaloids do activate myeloid cells via poorly defined mechanisms (23). Current experiments are focused on determining the mechanism of action of securinine and its array of effects on macrophages and other leukocytes.

As shown in this study, securinine or the securinine-like compounds may be effective adjuvant therapeutics. Securinine has been used extensively in vivo and levels greater than the amounts used above and has no obvious toxicity. In rodent studies, concentrations as high as 10 mg/kg or greater given i.p. are used to achieve the neuroprotective effects of securinine without any obvious toxicity (25). These results suggest that selective adjuvant activity can be obtained by using far lower concentrations. In this study, it was found that a single injection of 32 μg of securinine, which translates to about 1.28 mg/kg assuming a 25 g mouse, increases the clearance of C. burnetii in vivo.

In summary, it was shown that TLR agonists consistently increase macrophage activation and killing of phase II C. burnetii in vitro, but are inconsistent as adjuvant therapies for the bacterium in vivo under the conditions tested. In contrast, securinine and a number of securinine-like compounds that also induce macrophage activation and killing of C. burnetii in vitro consistently induce accelerated clearance of the bacterium in vivo. Because of the low toxicity of these compounds, securinine or securinine-like compounds may serve as effective immune adjuvants to increase non-specific innate resistance towards intra-cellular pathogens of macrophages.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.
1. Alam S., Laughton D. L., Walding A., Wolstenholme A. J. 2006. Human peripheral blood mononuclear cells express GABAa receptor subunits. Mol Immunol. 43:1432-1442.
2. Arricau-Bouvery N., Rodolakis A. 2005. Is Q Fever an emerging or re-emerging zoonosis? Vet. Res. 36: 327-349.
3. Beutler J. A., Karbon E. W., Brubaker A. N., Malik R., Curtis D. R., Enna S. J. 1985. Securinine alkaloids: a new class of GABA receptor antagonist. Brain Res. 135-140.
4. Brennan R. E., Samuel J. E. 2003. Evaluation of Coxiella burnetii antibiotic susceptibilities by real-time PCR assay. J Clin Microbiol. 41:1869-1874.
5. Brennan R. E., Russell K., Zhang G., Samuel J. E. 2004. Both inducible nitric oxide synthase and NADPH oxidase contribute to the control of virulent phase I Coxiella burnetii infections. Infect Immun. 72: 6666-6675.
6. Capo C., Moynault A., Collette Y., Olive D., Brown E. J., Raoult D., Mege J. L. 2003. Coxiella burnetii avoids macrophage phagocytosis by interfering with spatial distribution of complement receptor 3. J. Immunol. 170: 4217-4225.
7. Chebib M., Johnston G. A. 1999. The ‘ABC’ of GABA receptors: a brief review. Clin Exp Pharmacol Physiol. 26:937-940.
8. Fournier P., Marrie T. J., Raoult D. 1998. Diagnosis of Q fever. J Clin Microbiol. 36: 1823-1834.
9. Fraser I. P., Stuart L., Ezekowitz R. A. 2004. TLR-independent pattern recognition receptors and anti-inflammatory mechanisms. J Endotoxin Res. 10:120-124.
10. Froh M., Thruman R. G., Wheeler M. D. 2002. Molecular evidence for a glycine-gated chloride channel in macrophages and leukocytes. Am J Gastrointest Liver Physiol. 283:G856-G863.
11. Galvez-Ruano E., Aprison M. H., Robertson D. H., Lipkowitz K. B. 1995. Identifying agonistic and antagonistic mechanisms operative at the GABA receptor. J Neurosci Res. 42: 666-673.
12. Ghigo E., Capo C., Tung C. H., Raoult D., Gorvel J. P., Mege J. L. 2002. Coxiella burnetii survival in THP-1 monocytes involves the impairment of phagosome maturation: IFN-gamma mediates its restoration and bacterial killing. J. Immunol. 169: 4488-4495.
13. Hacker G., Redecke V., Hacker H. 2002. Activation of the immune system by bacterial CpG-DNA. Immunology. 105:245-251.
14. Hammerbeck D. M., Burleson G. R., Schuller C. J., Vasilakos J. P., Tomai M., Egging E., Cochran F. R., Woulfe S., Miller R. L. 2006. Administration of a dual toll-like receptor 7 and toll-like receptor 8 agonist protects against influenza in rats. Antiviral Res. Epub ahead of print.
15. Honstettre A., Ghigo E., Moynault A., Capo C., Toman R., Akira., Takeuchi O., Lepidi H., Raoult D., Mege J. L. 2004. Lipopolysaccharide from Coxiella burnetii is involved in bacterial phagocytosis, filamentous actin reorganization, and inflammatory responses through Toll-Like Receptor 4. J. Immunol. 172: 3695-3703.
16. Jutila M. A., Kroese F. G., Jutila K. L., Stall A. M., Fiering S., Herzenberg L. A., Berg E. L., Butcher E. C. Ly-6C is a monocytes/macrophage and endothelial cell differentiation antigen regulated by interferon-gamma. Eur J Immunol. 18: 1819-1826.
17. Lang G. Coxiellosis (Q fever) in animals. 1990. In: Marrie TJ, ed. Q fever, the disease. Boca Raton: CRC Press, 23-48.
18. Maurin, M., D. Raoult. 1999. Q Fever. Clin Microbiol Rev. 12: 518-553.
19. McQuiston J. H., Childs J. E. 2002. Q fever in humans and animals in the United States. Vector Borne Zoonotic Dis. 2: 179-191.
20. Pandey S., Agrawal D. K. 2006. Immunobiology of Toll-like receptors: emerging trends. Immunol Cell Biol. 84: 331-341.
21. Rezaei N. 2006. Therapeutic targeting of pattern-recognition receptors. International Immunopharmacology. 6:836-839.
22. Ribas A., Butterfield L. H., Glaspy J. A., Economou J. S. 2003 Current developments in cancer vaccines and cellular immunotherapy. J. Clin. Oncol. 21: 2415-2432.
23. Rios J. L., Redo M. C. 2005. Medicinal plants and antimicrobial activity. J Ethnopharmacol. 100: 80-84.
24. Stoker M. G., Marmion B. P. 1955. The spread of Q fever from animals to man. Bull World Health Organ. 13: 781-806.
25. Squires R. F., Saederup E. 1993. Indomethacin/ibuprofen-like anti-inflammatory agents selectively potentiate the gamma-aminobutyric acid-antagonistic effects of several norfloxacin-like quinolone antibacterial agents on [35S]t-butylbicyclophosphorothionate. Mol. Pharmacol. 43:795-800.
26. Tian J., Chau C., Hales T. G., Kaufman D. L. 1999. GABA(A) receptors mediate inhibition of T cell responses. J Neuroimmunol. 96:21-28.
27. Torres D., Barrier M., Bilf F., Quesniaux V. J., Maillet I., Akira S., Ryffel B., Erard F. 2004. Toll-like receptor 2 is required for optimal control of Listeria monocytogenes infection. Infect Immun. 72: 2131-2139.
28. Wheeler M. D., Thurman R. G. 1999. Production of superoxide and TNF-alpha from alveolar macrophages is blunted by glycine. Am J Physiol. 277:952-959.
29. Wheeler M., Stachlewitz R. F., Yamashina S., Ikejima K., Morrow A. L., Thurman R. G. 2000. Glycine-gated chloride channels in neutrophils attenuate calcium influx and superoxide production. FASAB J. 14:476-484.
30. Wilson E., Aydintug M. K., Jutila M. A. 1999. A circulating bovine □□ T cell subset, which is found in large numbers in the spleen, accumulates inefficiently in an artificial site of inflammation: correlation with lack of expression of E-selectin ligands and L-selectin. J. Immunol. 162: 4914-4919.
31. Woldehiwet Z. 2004. Q fever (coxiellosis): epidemiology and pathogenesis. Res Vet Sci. 77: 93-100.
32. Zamboni D. S., Campos M. A., Torrecilhas A. C. T., Kiss K., Samuel J. E., Golenbock D. T., Lauw F. N., Roy C. R., Almeida I. C., Gazzinelli R. T. 2004. Stimulation of Toll-like Receptor 2 by Coxiella burnetii is required for macrophage production of pro-inflammatory cytokines and resistance to infection. Jour Biol Chem. 24: 54405-54415.
33. Zamboni D. S., Mortara R. A., Rabinovitch M. 2001. Infection of Vero cells with Coxiella burnetii phase II: relative intracellular bacterial load and distribution estimated by confocal laser scanning microscopy and morphometry. J Microbiol Methods. 43: 223-32.

1. A method of activating macrophages in a subject in need thereof by administering to said subject a pharmaceutical composition comprising the general formula (I): 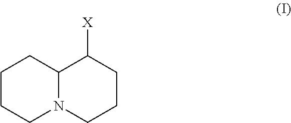
wherein said X is —NR¹R², —CH₂—NH—C(O)—R³, —CH₂—O—C(O)—R⁴, or —CH₂—OR⁵; and
wherein
R¹ and R², taken together with the nitrogen atom to which they are shown both attached, form piperidine-2,6-dione, pyrrolidine-2,5-dione, or isoindoline-1,3-dione;
R³ is straight or branched alkyl of 1 to 6 carbon atoms;
R⁴ is straight or branched alkyl of 1 to 6 carbon atoms, which is unsubstituted or substituted with hydroxyl; and
R⁵ is hydrogen, sodium, cyclic alkyl of 5 to 7 carbon atoms, or pyrrolidine-2,5-dione.

2. The method of claim 1, wherein said subject is infected with an intracellular microbe.

3. The method of claim 1, wherein said microbe is selected from the group consisting of bacteria, viruses and parasites.

4. The method of claim 3, wherein said bacteria are Coxiella burnetii.

5. The method of claim 1, wherein said pharmaceutical composition comprises at least one TLR agonist.

6. The method of claim 1, wherein said pharmaceutical composition comprises at least one antibiotic.

7. A method of preventing, treating or ameliorating an infectious disease comprising administering securinine to a subject.

8. The method of claim 7, wherein said infectious disease is caused by a bacterial infection.

9. The method of claim 8, wherein said bacterial infection caused by a bacteria able to multiply inside a eukaryotic cell.

10. The method of claim 9, wherein said bacteria that is able to multiply inside a eukaryotic cell is selected from the group consisting of Salmonella enterica serovar typhimurium, Legionella pneumophila, Coxiella burnettii, Francisella tularensis, Mycobacterium tuberculosis, obligate intracellular Chlamydia spp., Listeria monocytogenes, Shigella flexneri, enteroinvasive E. coli and Rickettsia.

11. The method of claim 10, wherein said bacteria are Coxiella burnetii.

12. The method of claim 7, wherein said infectious disease is caused by a virus.

13. The method of claim 12, wherein said virus is selected from the group consisting of influenza, corona virus, hepatitis viruses, human immunodeficiency virus, herpes and respiratory syncytial virus.

14. The method of claim 7, wherein said infectious disease is caused by a parasite.

15. The method of claim 14, wherein said parasite is Leishmania tropica, Trypanosoma brucei, Toxoplasma gondii, Schistosoma haematobium and Plasmodium falciparium.

16. The method of claim 7, wherein said subject is a human.

17. The method of claim 7, wherein the securinine is administered with an additional compound.

18. The method of claim 17, wherein said additional compound is an antibiotic.

19. The method of claim 18, wherein said antibiotic is selected from the group consisting of aminoglycosides, carbapenems, chloramphenicol, fluoroquinolones, glycopeptides, lincosamides, macrolides/ketolides, cephalosporins, monobactams, penicillins, and tetracyclines.

20. The method of claim 17, wherein said additional compound is a TLR agonist.

21. The method of claim 20, wherein said TLR agonist agonizes TLR-2 and/or TLR-4.

22. The method of claim 21, wherein said TLR agonist is selected from the group consisting of lipoteichoic acid, petidoglycan, and lipopolysaccharide.

23. The method of claim 7, wherein said securinine is administered to said subject orally, intradermally, intranasally, intramusclarly, intraperitoneally, intravenously, or subcutaneously.

24. A method of activating macrophages in a subject in need thereof, comprising administering to said subject a pharmaceutical composition comprising securinine.

25. The method of claim 24, wherein said subject is infected with an intracellular microbe.

26. The method of claim 25, wherein said microbe is selected from the group consisting of bacteria, virus and parasite.

27. The method of claim 26 wherein said bacteria are Coxiella burnetii.

28. The method of claim 24, wherein said pharmaceutical composition comprises at least one additional TLR agonist.

29. The method of claim 24, wherein said pharmaceutical composition comprises at least one antibiotic.

30. The method of claim 24, wherein said pharmaceutical composition comprises at least a compound comprising formula (I).

31. The method of claim 24, wherein said securinine is administered to said subject orally, intradermally, intranasally, intramusclarly, intraperitoneally, intravenously, or subcutaneously.


SEQUENCE LISTING (2 sequences)

SEQ ID NO: 1 - DNA, 21 bp, Coxiella burnetii
cgcgttcgtcaaatccaaata

SEQ ID NO: 2 - DNA, 20 bp, Coxiella burnetii
gacgccttccatttccaaaa